(19) United States
(12) Patent Application Publication
(10) Pub. No.: US 2010/0008950 A1
(43) Pub. Date: Jan. 14, 2010

(54) COMPOSITION AND METHOD FOR THE INDUCTION OF IMMUNITY AGAINST BACILLUS CEREUS GROUP BACTERIA

(76) Inventors: Timothy D. Read, Atlanta, GA (US); Sanghamitra Mukhopadhyay, Rockville, MD (US)

Correspondence Address: NAVAL MEDICAL RESEARCH CENTER;ATTN: (CODE 00L), 503 ROBERT GRANT AVENUE, SILVER SPRING, MD, 20910-7500, US

(21) Appl. No.: 12/501,021

(22) Filed: Jul. 10, 2009

Related U.S. Application Data
(60) Provisional application No. 61/079,535, filed on Jul. 10, 2008.

(51) Int. Cl. A61K 39/07; G01N 33/569; C40B 30/04; C12Q 1/68

(52) U.S. Cl. 424/200.1; 424/246.1; 424/201.1; 435/7.32; 506/9; 435/6

(57) ABSTRACT

A composition comprised of one or more bacterial proteins that are conserved among members of Bacillus cereus that is capable of inducing an immune response against members of Bacillus cereus group bacteria. The invention also relates to a method of utilizing the composition to induce an immune response.

### CROSS-REFERENCE OF RELATED APPLICATIONS

This application claims the benefit of U.S. Provisional Application No. 61/079,535, filed 10 Jun. 2008.

### BACKGROUND OF THE INVENTION

1. Field of the Invention

The current invention relates to a composition for detecting Bacillus cereus group bacteria and for inducing an immune response against said bacteria and methods thereof.

2. Description of the Related Art

Pathology due to B. anthracis infection is primarily due to the release by the organism of protective antigen (“PA”) in association with lethal factor (“LF”) and edema factor (“EF”) (1). The complete DNA and protein sequence of PA has been published and its three-dimensional structure is known from x-ray crystallography (2). The characteristics and biological functions of the four domains of PA are also available permitting selection of epitopes within the domains based on antigenic properties (2-6).

In animal studies, as well as studies of natural human infection, it was shown that individuals who survived an infection produced antibodies to PA, suggesting its importance in the protection (7).

All Department of Defense personnel are vaccinated against anthrax before deploying to at-risk areas of operation. Furthermore, research personnel working with infectious strains of anthrax are also vaccinated, prophylactically. The current human anthrax vaccine (adsorbed) (“AVA”) licensed in the United States consists mostly of PA from an attenuated, non-encapsulated strain of B. anthracis and aluminum hydroxide adjuvant (8, 9), which is known to protect against challenge with spores of wild type B. anthracis. Additional targets for B. anthracis vaccines have been other proteins in the tripartite toxin (encoded by genes on plasmid pXO1) and the poly-γ-D-glutamic acid capsule (on plastic pXO2). Screens for protective immunogens on the chromosome have focused narrowly on targets specific to the sequenced B. anthracis Ames strain [10].

While many vaccines developed against virulence gene-specific targets offer excellent protection against infection, biological weapons (“BW”) with enhanced virulence and ability to evade current medical countermeasures may be created by cloning or altering genes, such as toxins. Genomic studies revealed that close relatives of B. anthracis have very similar composition of chromosomal genes [11]. Thus, there is a potential for the transfer of toxins and capsule between these genetic backgrounds to create new pathogens. In addition, some members of B. cereus sensu lato (s. l.) may be able, in rare cases, to cause inhalational disease in humans or primates. “Bacillus cereus group” is the term given to the collection of species, which includes B. anthracis, B. thuringiensis, B. cereus, B. mycoides, B. weihenstepphanensis and B. pseudomycoides. Example of such an organism is B. cereus G9241, which carries a pXO1 plasmid and lethal toxin genes that is almost identical to B. anthracis [11]. Therefore, it is important to develop vaccine compositions and detection methods effective against a broad range of related organisms that could serve as hosts for genetically modified toxins and virulence factors. Identification of conserved immunogenic proteins among the Bacillus cereus group is the important first step.

### SUMMARY OF INVENTION

The current invention relates to a composition and method of immunizing against bacteria within the Bacillus cereus group. It also relates to method of detecting bacteria within the Bacillus cereus group.

An object of the invention is an immunogenic composition consisting of one or more of 18 selected bacterial proteins. Each of the 18 selected bacterial proteins contains a polypeptide sequence that exhibits high homology within the B. cereus group of bacteria.

Another object of the invention is a method of inducing an immune response against Bacillus anthracis and other members of the Bacillus cereus group of bacteria by administering a composition comprising a one or more of 18 selected bacterial proteins exhibiting high homology within the B. cereus group.

Yet another object of the invention is a method of detecting Bacillus cereus group of bacteria using a one or more of 18 selected bacterial proteins exhibiting high homology within the B. cereus group. One method for using the conserved proteins for detection includes raising antibodies against one or more of the proteins (either isolated directly from the bacterium or through recombinant technology such as phage display) and attaching a reporter ligand such as a fluorophore. Another method would be to select one or more subsequences (epitopes) of one or more of the selected proteins and raise antibodies. Another method would be to use other molecules that specifically bind one or more of the 18 proteins such as nucleotide aptamers and/or binding proteins and couple these with reporter molecules such as fluorophores.

### BRIEF DESCRIPTION OF THE DRAWINGS

FIG. 1. Illustration of overall scheme of genomic analysis.

FIG. 2. Filtering strategy for down selection of spore-associated conserved ORFs from B. cereus s. l.

FIG. 3. Proteins identified by genomic analysis.

FIG. 4. Phylogeny of B. cereus s. l. genomes

FIG. 5. Illustration of overall scheme of proteomic analysis.

FIG. 6. Western blot analysis of spore proteins of B. anthracis and closely related strains against rabbit immune sera generated against B. anthracis.

### DETAILED DESCRIPTION OF PREFERRED EMBODIMENTS

The invention contemplates the construction of an immunogenic formulation against species of B. cereus comprising a number of homologous and immunogenic proteins from related but functionally diverse bacteria of Bacillus cereus. The formulation would be capable of inducing a response against Bacillus spore forming bacteria including Bacillus cereus bacteria that are closely related to B. anthracis but that are not normally pathogenic unless they have been naturally or artificially transfected with toxins or virulence factors.

### EXAMPLE 1

### Identification of Proteins

One commonality of any B. cereus sensu lato (s.l.) derived biothreat pathogen is that it would cause disease via spore inhalation. Therefore, a vaccine developed based on a number of conserved spore proteins from B. cereus s. l. could add to the arsenal of protection options. Two high-throughput approaches: genome-based bioinformatic analysis and comparative proteomic analysis are used to study spores of B. cereus s. l. in order to select conserved spore protein targets.

Proteins of B. cereus group bacteria that were either genetically or physically disparate or similar were identified by simultaneous genome-based bioinformatic analysis and comparative proteomic analysis of B. cereus s. l. spore proteins. The goal of the analytic method was to determine if a particular protein was separable from its counterpart within another member of the same bacterial group or whether there was close genetic and physical similarity for that protein within the group.

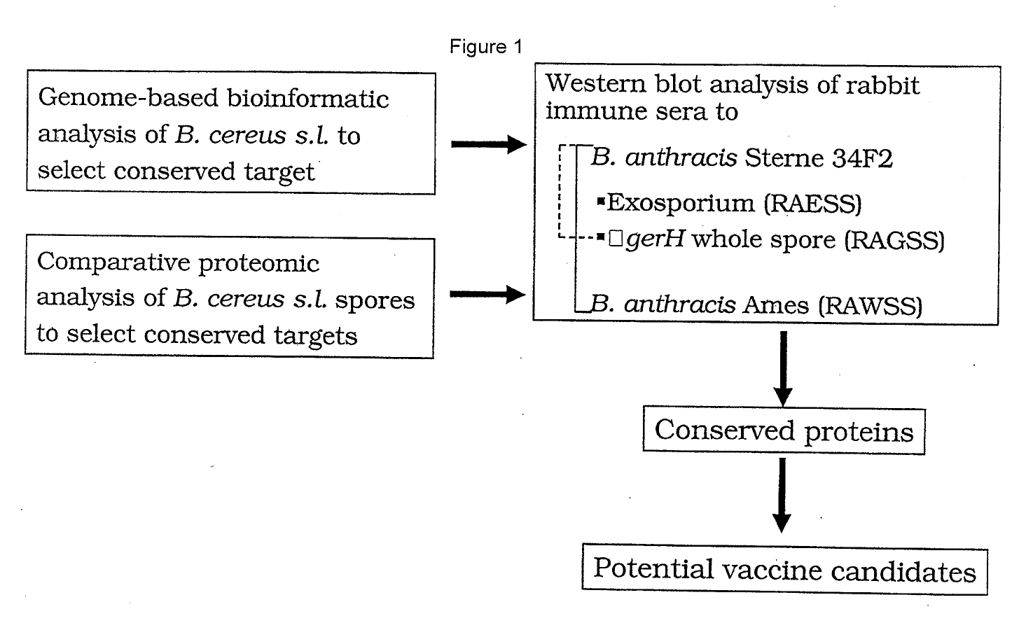

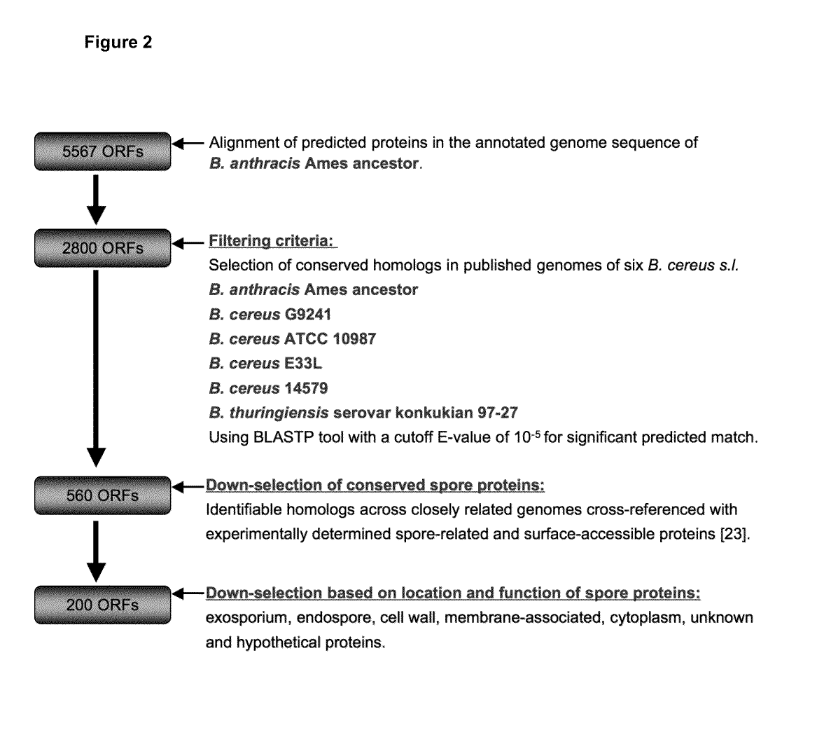

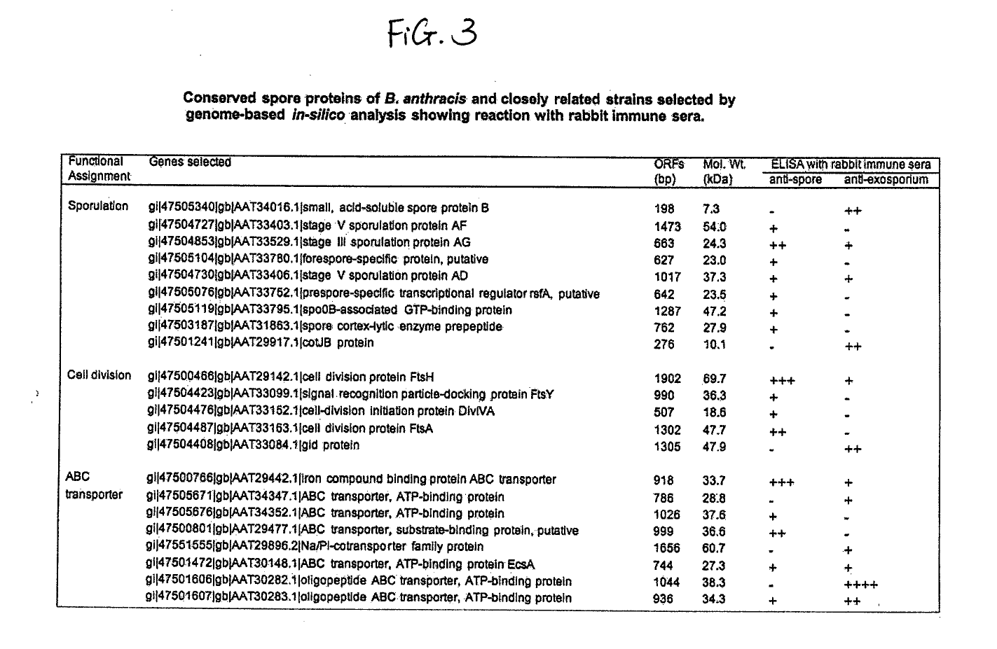

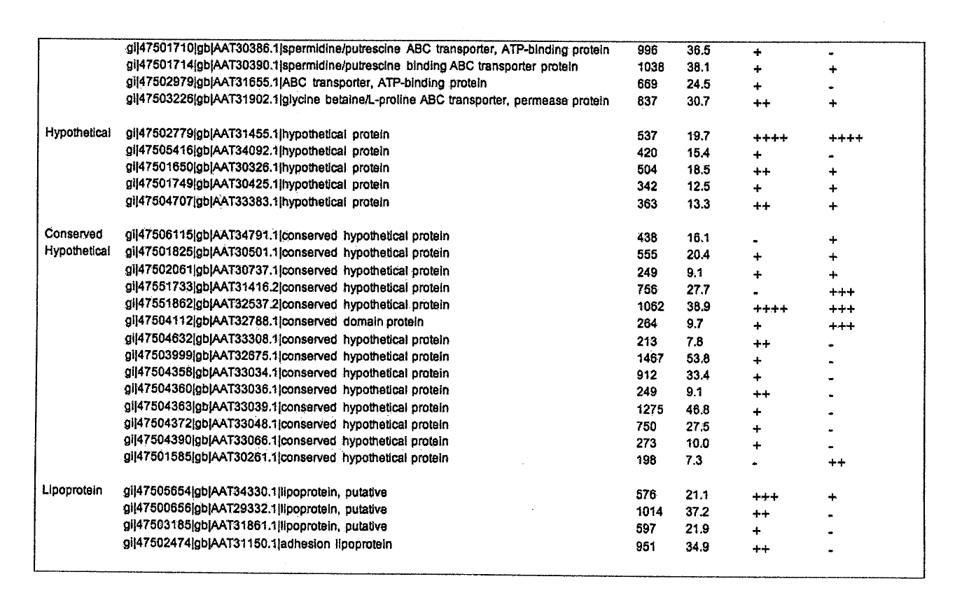

In order to find conserved proteins the 5,567 predicted proteins in the annotated genome sequence B. anthracis Ames ancestor (NC_{—}007530, NC_{—}007323, NC_{—}007322) are aligned against a database of the predicted proteomes of published genomes from B. anthracis (Ames); B. cereus (G9241); B. cereus (10987); B. cereus (E33L); B. cereus (14579); B. thuringiensis serovar konkukian (97-27) using BLASTP (Basic Local Alignment Search Tool). Scoring was predicated on identifiable homologues, localized in surface accessible manner, and also predicated on function, specifically involved in sporulation. Using a cutoff E-value of 10⁻⁵ for the significance of the predicted match, 2800 proteins were found conserved in all six B. cereus group genomes. The proteins are then cross-referenced against more than 750 endo- and exosporium constituent proteins of B. anthracis identified by multidimensional chromatography and tandem mass spectroscopy and obtained open reading frames (“ORFs”) of 560 proteins. Of the 560 ORFs, 200 ORFs were selected for in vitro gene specific expression and purification using transcriptionally active PCR fragment system. This is selection scheme is illustrated in FIG. 2. One-hundred ninety two (192) proteins were successfully synthesized. Using the selection criteria outlined in FIG. 1, 75 initial immunogenic proteins were identified. As identified in FIG. 3, 23 proteins reacted with both Rabbit Anti-GerH whole Spore Sera (“RAGSS”) and Rabbit anti-ExoSporium Sera (“RAESS”). Thirty-seven (37) proteins reacted with sera generated from RAGSS suggesting these proteins are present either in the spore coat, cortex and/or in the core. Fifteen (15) proteins reacted with RAESS with known functions. There are 3 proteins with undetermined functions also reacted with RAESS.

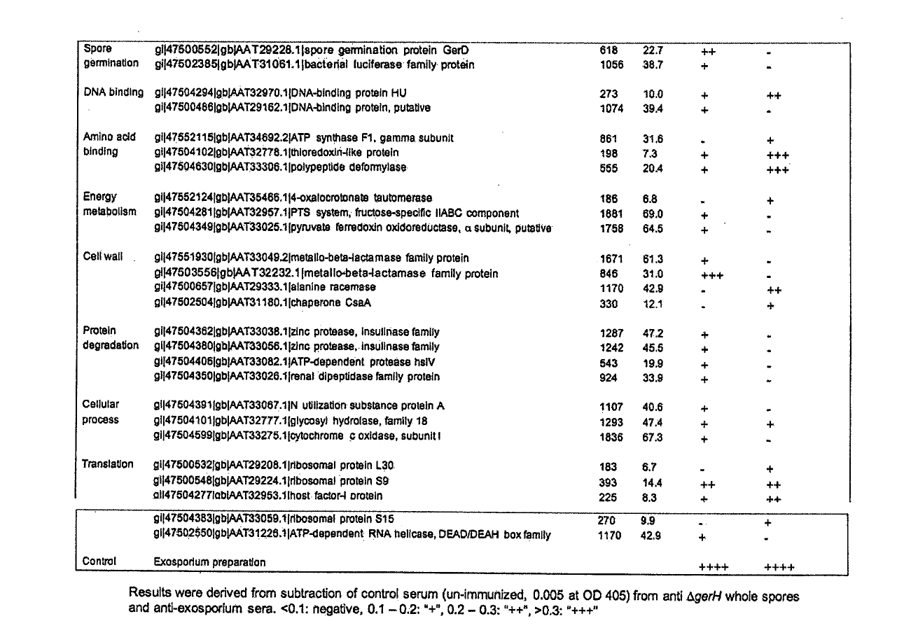

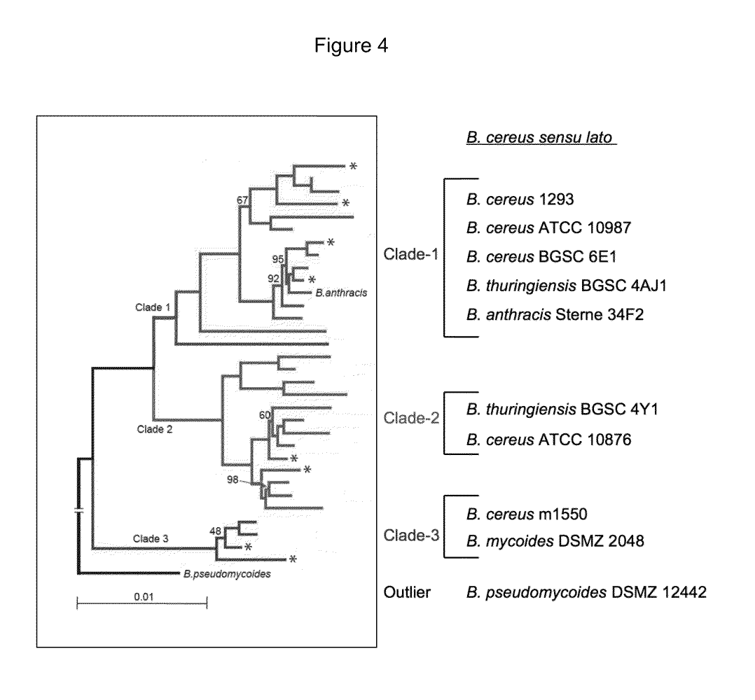

Simultaneous to genomic analysis, proteomic analysis was conducted against related, but functionally diverse members of B. cereus s. l. group, illustrated in FIG. 4. Further electron-microscopic ultrastructural analysis of these groups of bacteria indicates that structural differences are observed between these highly related organisms.

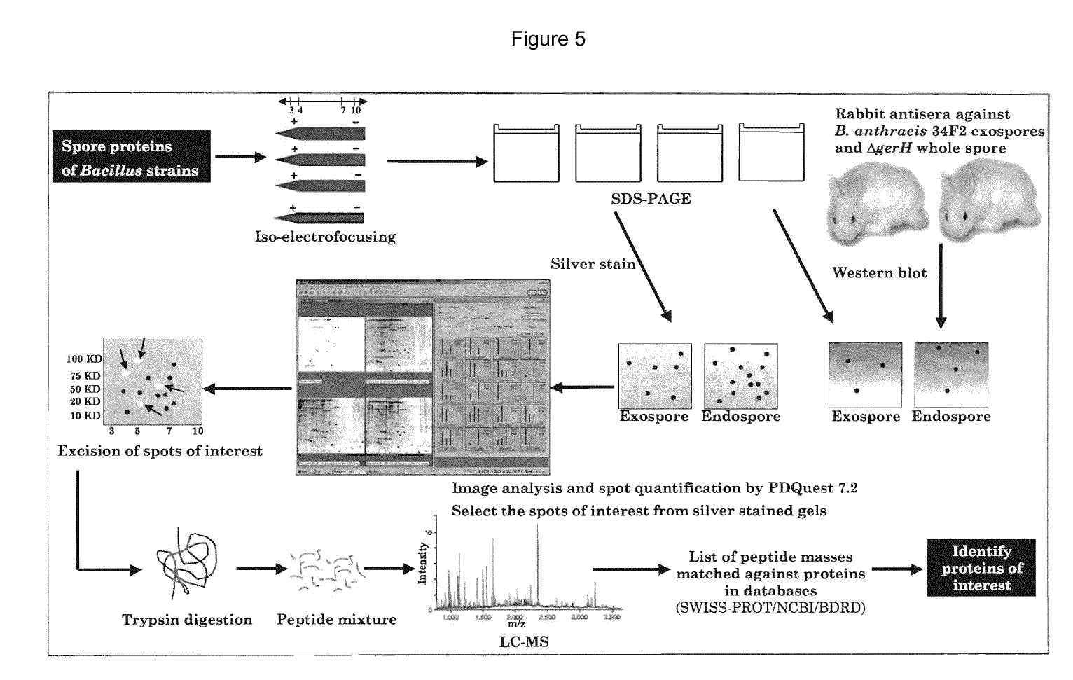

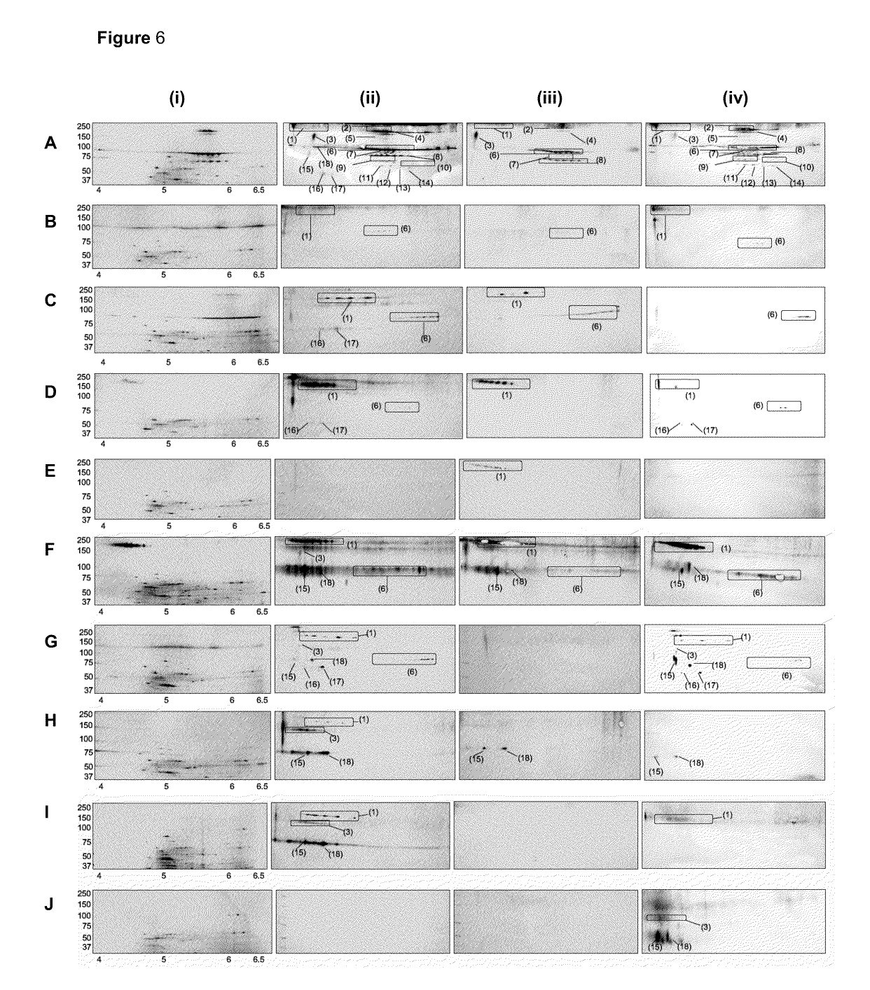

Proteomic analysis methodology was generally conducted as outlined in FIG. 5. As illustrated in FIG. 5, spore proteins from Bacillus strains were compared based on isoelectric focusing migration, sizing in SDS-polyacrylamide gel electrophoresis (SDS-PAGE) and recognition by sera from rabbits. The sera was raised in rabbits that had either been immunized with either with B. anthracis sterne strain (34F2) exospore or whole B. anthracis spores (B. anthracis ΔgerH), which is deficient in its capability of germinating and therefore can be used to immunize animals without pathogenicity. The rate of overall match in the gels is shown in Table 1. The proteomics analysis yield a group of 18 proteins that exhibited a strong signal in western blot analysis using sera from rabbits immunized with wither B. anthracis 34F2 exospore or B. anthracis 34F2 (ΔgerH). These are illustrated in Table 2. Of the 18 proteins, 7 exhibited a significant level of physical conservation. As shown in FIG. 6, all of the 18 proteins were detected in Western immunoblot of RAGSS (FIG. 6A, panel (ii)), and 14 of the sports were detected against Rabbit Anti-Whole Spore (un-germinated) Sera (RAWSS) (FIG. 6A, panel (iv)). Additionally, six proteins were sera-reactive in most of the Bacillus strains, including conserved repeat domain protein (Crd), S-layer protein EA1 precursor (Eag), translation elongation factor G (FusA), Molecular Chaperone (DnaK), Chaperonin (GroEL) and Translation elongation factor Tu (Tuf), see Table 3.

TABLE 1
Matching summary of protein spots expressed in two-dimensional
electrophoretic maps of spores of B. anthracis and closely related strains.

| | | | | Match |
|---|---|---|---|---|
| | | Spot | Spots | rate |
| Strains | Clades | count | matched | (%) |
| B. anthracis Sterne 34F2 | 1 | 370 | 370 | 100 |
| B. thuringiensis BGSC 4AJ1 | 1 | 390 | 322 | 83 |
| B. cereus 1293 | 1 | 337 | 269 | 80 |
| B. cereus BGSC 6E1 | 1 | 255 | 212 | 83 |
| B. cereus ATCC 10987 | 1 | 231 | 190 | 82 |
| B. cereus ATCC 10876 | 2 | 236 | 184 | 78 |
| B. thuringiensis BGSC 4Y1 | 2 | 294 | 229 | 78 |
| B. cereus m1550 | 3 | 228 | 185 | 81 |
| B. mycoides DSMZ 2048 | 3 | 378 | 298 | 79 |
| B. pseudomycoides DSMZ 12442 | Outlier | 246 | 184 | 75 |

Spots matched to every gel - 72%

TABLE 2
Spore Proteins identified in B. anthracis and closely related stains from Western blot
analysis against rabbit immune sera.

| Spot | | | | Theoritical |
|---|---|---|---|---|
| no. | Locus tag | Accession no. | Protein | Mw/pl |
| 1 | GBAA3601 | gi47528887 | Conserved repeat domain protein (Crd) | 245.09/4.17 |
| 2 | GBAA1094 | gi47526368 | Wall-associated protein, putative (RhsA) | 249.01/5.97 |
| 3 | GBAA3677 | gi47528961 | Aconitate hydratase 1 (AcnA) | 99.01/4.8 |
| 4 | GBAA0052 | gi47525306 | Trasncription-repair coupling factor (Mfd) | 134.15/5.36 |
| 5 | GBAA4157 | gi47529455 | Pyruvate decarboxylase (Pyc) | 128.57/6.0 |
| 6 | GBAA0887 | gi47526173 | S-layer protein EA1 precursor (Eag) | 91.36/5.70 |
| 7 | GBAA3944 | gi47529234 | Polyribonucleotide nucleotidyltransferase (Pnp) | 78.20/5.16 |
| 8 | GBAA4843 | gi47530138 | Pyruvate kinase (PykA-2) | 62.10/5.20 |
| 9 | GBAA5583 | gi47530904 | CTP synthase (CtrA) | 59.75/5.28 |
| 10 | GBAA0309 | gi47500720 | Δ-1-pyrroline-5 carboxylate dehydrogenase | 56.22/5.43 |
| 11 | GBAA3609 | gi47528895 | Aldehyde dehydrogenase (DhaS) | 53.74/5.4 |
| 12 | GBAA4184 | gi47529480 | Pyruvate dehydrogenase, E1α (PdhA) | 41.44/5.52 |
| 13 | GBAA0252 | gi47525509 | Alanine racemase (Dal-1/Air) | 43.66/5.51 |
| 14 | GBAA5222 | gi47505676 | ABC transporter, ATP binding protein (MetN) | 37.26/6.18 |
| 15 | GBAA4539 | gi47529836 | Molecular chaperone DnaK | 65.76/4.65 |
| 16 | GBAA0267 | gi47525527 | Chaperonin GroEL | 57.43/4.79 |
| 17 | GBAA0108 | gi47525364 | Translation elongation factor Tu (Tuf) | 42.93/4.93 |
| 18 | GBAA0107 | gi47525363 | Translation elongation factor G (FusA) | 76.33/4.91 |

The spot number indicates the corresponding protein spot in the 2D electrophoretic Western blots shown in FIG. 6.

TABLE 3
Sera-reactive Conserved Bacillus Spore Proteins

| Spot | Locus | | Theoritical | Ba | Bt | Bc | Bc | Bc | Bc | Bt | Bc | Bm | Bp |
|---|---|---|---|---|---|---|---|---|---|---|---|---|---|
| no.* | (GBAA) | Protein | Mw/pl | 34F2 | 4AJ1 | 1293 | 6E1 | 10987 | 10876 | 4Y1 | 1550 | 2048 | 12442 |
| 1 | 3601 | Conserved repeat domain | 245.09/4.17 | +•▴ | +▴ | +• | +•▴ | • | +•▴ | +▴ | + | +▴ | |
| | | protein (Crd) | | | | | | | | | | | |
| 2 | 1094 | Wall-associated protein, | 249.01/5.97 | +•▴ | | | | | | | | | |
| | | putative (RhsA) | | | | | | | | | | | |
| 3 | 3677 | Aconitate hydratase 1 (AcnA) | 99.01/4.8 | +•▴ | | | | | + | +▴ | + | + | ▴ |
| 4 | 0052 | Trasncription-repair coupling | 134.15/5.36 | +•▴ | | | | | | | | | |
| | | factor (Mfd) | | | | | | | | | | | |
| 5 | 4157 | Pyruvate decarboxylase (Pyc) | 128.57/6.0 | +▴ | | | | | | | | | |
| 6 | 0887 | S-layer protein EA1 precursor (Eag) | 91.36/5.70 | +•▴ | +•▴ | +•▴ | +▴ | | +•▴ | +▴ | | | |
| 7 | 3944 | Polyribonucleotide | 78.20/5.16 | +•▴ | | | | | | | | | |
| | | nucleotidyltransferase (Pnp) | | | | | | | | | | | |
| 8 | 4843 | Pyruvate kinase (PykA-2) | 62.10/5.20 | +•▴ | | | | | | | | | |
| 9 | 5583 | CTP synthase (CtrA) | 59.75/5.28 | +▴ | | | | | | | | | |
| 10 | 0309 | Δ-1-pyrroline-5 carboxylate | 56.22/5.43 | +▴ | | | | | | | | | |
| | | dehydrogenase | | | | | | | | | | | |
| 11 | 3609 | Aldehyde dehydrogenase (DhaS) | 53.74/5.4 | +▴ | | | | | | | | | |
| 12 | 4184 | Pyruvate dehydrogenase, | 41.44/5.52 | +▴ | | | | | | | | | |
| | | E1α (PdhA) | | | | | | | | | | | |
| 13 | 0252 | Alanine racemase (Dal-1/Alr) | 43.66/5.51 | +▴ | | | | | | | | | |
| 14 | 5222 | ABC transporter, ATP binding | 37.26/6.18 | +▴ | | | | | | | | | |
| | | protein (MetN) | | | | | | | | | | | |
| 15 | 4539 | Molecular chaperone DnaK | 65.76/4.65 | + | | + | +▴ | | +•▴ | +▴ | +•▴ | + | ▴ |
| 16 | 0267 | Chaperonin GroEL | 57.43/4.79 | + | | | | | | +▴ | | | |
| 17 | 0108 | Translation elongation factor | 42.93/4.93 | + | | | | | | | | | |
| | | Tu (Tuf) | | | | | | | | | | | |
| 18 | 0107 | Translation elongation | 76.33/4.91 | + | | + | +▴ | | +•▴ | | +•▴ | + | ▴ |
| | | factor G (FusA) | | | | | | | | | | | |

+spots present in Western immunoblot against anti- B. anthracis 34F2 ΔgerH spore
•spots present in Western immunoblot against anti- B. anthracis 34F2 exospore
▴spots present in Western immunoblot against anti- whole spore (irradiated un-germinated B. anthracis Ames)
*The spot number indicates the corresponding protein spot in the 2D electrophoretic map in FIG. 5

### EXAMPLE 2

### Composition and Method of Using Composition as Immunogen Against B. cereus Group

The proteins identified in Example 1, despite being from functionally diverse species within the B. cereus group, have either high genomic conservation or exhibit physical similarity based on migration under isoelectric focusing conditions, SDS-PAGE or reactivity to immune serum. An immunogenic composition can, therefore, be composed of one or more of these proteins or immunogenic fragments, thereof. The composition can be administered parentally, subcutaneously, transdermally, intramuscularly, orally, and transcutaneously.

The immunogenic composition, therefore would be composed on one or more of the proteins selected from the group consisting of cell surface protein, gram positive anchor (Dufl1), conserved repeat domain protein (Crd), transcription-repair coupling factor (Mfd), pyruvate decarboxylase (Pyc), predicted exporter (HP), aconitate hydratase 1 (AcnA), S-layer protein EA1 precursor (Eag), polyribonucleotide nucleotidyl transferase (Pnp), translation elonation factor G (FusA), pyruvate kinase (PykA2), CTP synthase (CtrA), chaperonin, 60 kDa (GroEL), Δ-1-pyrroline-5 carboxylate dehydrogenase, aldehyde dehydrogenase (DhasS), alanine racemase (Dal-1), phosphoglycerate kinase (Pgk), pyruvate dehydrogenase complex E1 component, alpha subunit (PdhA), and ABC transporter, ATP binding protein (MetN).

### EXAMPLE 3

### Identification of Proteins from Functionally Diverse Species within a Group

The methodology taught in Example 1 can be used to more rapidly and definitively predict proteins or polypeptides that would be useful as antigens in identification or diagnostic assays for bacterial or viral organisms.

In the method, proteins exhibiting different migration or serum reactivity patterns can be specifically compared to standard proteins from a member of the bacterial group to which it is known to be a member. The physical characteristics of the protein, as outlined by genomic and proteomic analysis, as in Example 1, would therefore identify the protein as belonging to a specific species within the bacterial group.

Alternatively, the method of Example 1 can be used to quickly identify proteins from bacteria within a group that have identifiable sequence differences. These identified proteins would therefore be subjected to sequence analysis in order to identify specific sequence regional differences within the protein, which can be used to develop PCR or other antibody based assays to these regions.

### EXAMPLE 4

### Prophetic Method for the Identification of Bacillus cereus Group Bacteria

The proteins identified in Example 1 can be utilized in antibody or molecular methods for the detection of Bacillus cereus group bacteria. The polypeptide regions that are conserved within the members of B. cereus can be used as antigens in enzyme-linked immunosorbent assays (ELISA) or immobilized onto microchips for the detection of sera from potentially B. cereus exposed patients. Alternatively, sera can be raised to these targets, which can be used to detect B. cereus group bacteria for use in antibody-based assays for the detection and identification of B. cereus group bacteria.

Alternatively, polynucleotides encoding fragments of the 18 proteins, especially regions that are conserved among B. cereus, can be used in molecular assays such as polymerase chain reaction or hybridization reactions. A prophetic example of an assays includes the steps:
a. Amplify DNA from bacteria in a sample by PCR reaction using a template from one or more of the 18 proteins. Preferably, the region of each protein that is amplified is a region conserved among members of B. cereus.
b. Detect and measure the amplified products. Detection and measurement of amplified products can be by any means including exposing the amplified DNA to a panel, e.g. immobilized on a microchip, of polynucleotide sequences encoding the entire or fragments of one or more of the 18 proteins.

### EXAMPLE 5

### Prophetic Vaccine Challenge Studies

### Selection of the Potential Vaccine Combination Candidate Production and Purification

Seven potential vaccine candidates are produced in bulk using high-throughput GTS cell free TAP expression system followed by the RTS® ProteoMaster (Roche Applied Science, Indianapolis, Ind.) based on the target genes of conserved repeat domain protein, aconitate hydratase, S-layer protein EA1 presursor, alanine racemase, ABC transporter/ATP binding protein, molecular chaperson DnaK and translational elongation factor. The proteins are then purified for immunization and validation studies. Before immunization, each protein is tested for capability to produce antibodies in A/J mice.

### Immunization

A/J mice (6-8 week old) are obtained from Jackson laboratories (Bar Harbor, Me.) and maintained under specific-pathogen free conditions with free access to food and water. The seven purified proteins are administered in combination to the mice intramuscularly or intraperitoneally using the alum adjuvant system at various doses with adjuvant prepared to an equivalent concentration. Varying vaccine doses provides for the identification of an optimum dosage required to generate immunogenic reaction in mice. For all immunization combinations, the mice are follow the same immunization regimen of priming on day 0 and boosting on day 14 and day 28. Serum samples will be drawn at a two-week interval (up to 56 days) and on the days of immunization to measure antibody responses against the potential vaccine candidates. At the end of the study, splenocytes will be harvested to examine cell-mediated responses to these vaccines.

### ELISA for A/J Mice Serum Antibody

In order to analyze the hormonal immune response to the TAP generated proteins (potential vaccine candidates), serum antibody titers to the seven proteins combinations will be assayed by ELISA, by using each of the protein combinations as capture antigen. The data will be presented as geometric mean titers with standard deviation (SD).

### ELISPOT Assays for Cytokine Production

In order to analyze the cell-mediated immune response to the TAP generated protein candidates, secretion of interleukin-4 (IL-4) and gamma interferon (IFN-γ) by spleen cells from naive and immunized A/J mice will be examined by ELISPOT assay (eBD™ Biosciences, San Jose, Calif.). After the immunization schedule, the mice will be culled by cervical dislocation and the spleens will be removed and forced through disposable 70 mm cell strainers (BD Biosciences, San Jose, Calif.) to obtain single-cell suspensions. Following centrifugation to pellet the cells, red blood cells will be removed by using lysing buffer (Sigma, USA). The remaining cells are washed, counted, and seeded onto ELISPOT plates in medium containing each of the seven candidates respectively. Four replicates are be plated for each of the seven proteins per treatment group. Concanavalin A (Sigma, USA) is used as a positive control. After an overnight incubation of the ELISPOT plates at 37° C. in the presence of 5% CO₂ in a humidified incubator, assay development will be performed according to the kit manufacturer's instructions. The data will be presented as mean values with SD.

### Validation of the Potential Vaccine Candidates by Mouse Challenge

After analyzing the data from ELISA and ELISPOT assays, the best combination of the potential vaccine candidates will be selected for challenge study in A/J mice based on their immunogenicity and functionality.

### Challenge Study

A/J mice will be immunized with the potential vaccine candidate, challenged with spores of B. anthracis Sterne 34F2, B. anthracis Ames and B. cereus G9241 strains and survival rates monitored.

The growth of and challenge with the selected clinical Bacillus strains will be performed in the BSL-3 laboratory conditions at NMRC. Bacillus strains will be grown in brain heart infusion broth overnight at 37° C. in a shaking condition and diluted to give an estimated challenge dose of 10⁴ spores per mouse. A/J mice will be challenged by the intraperitoneal route on day 35 following primary immunization with the potential vaccine candidate (along with the adjuvant, alum) and observed for 42 days post-challenge, at which point any survivors will be culled. Depending on the mice survival rates, the vaccine candidate(s) will be selected for further development.

### Assessment of Bacterial Load

The challenge survivors will be assessed for bacterial load by culture of brain, liver, spleen and blood. Organs will be processed by passing through 70 μm nylon sieves into sterile PBS. Blood will be obtained by cardiac puncture and diluted in sterile PBS. Samples will be inoculated onto blood agar plates, incubated overnight at 37° C. and examined for the presence or absence of the selected B. anthracis Sterne 34F2, B. anthracis Ames and B. cereus G9241 strains used for the mouse challenge.

### PROPHETIC EXAMPLE 6

### Diagnostic Method to Distinguish Between Bacillus and Other Bacteria

### Generation of Rabbit Immune Sera

Rabbit immune sera will be generated against each of the seven proteins (conserved repeat domain protein, aconitate hydratase, S-layer protein EA1 precursor, alanine racemase, ABC transporter/ATP binding protein, molecular chaperon DnaK and translation elongation factor). Two out-bred rabbits will be immunized with each antigen via intra-dermal injection. To generate immune sera, each rabbit will receive 100 μg of purified protein. Nine boosts per antigen will be given to each rabbit, i.e. biweekly boost schedule for the first 6 months, followed by monthly boost the next 3 months. The immune sera obtained from the two rabbits in each of the groups, against seven proteins will be pooled respectively, for ELISA. Anti-sera from un-immunized rabbits will be collected as controls, the control rabbits will be injected with phosphate buffered saline.

### Western Blot Analysis of Bacterial Proteins

Proteins from 15 different bacterial cell lysate will be run on polyacrylamide gel electrophoresis and transferred to polyvinylidene difluoride (PVDF) membranes using Towbin buffer (25 mM trizma base, 190 mM glycine, 20% methanol) at 100 V for 30 min. After the transfer, the PVDF membrane will be blocked with 3% skim milk prepared in phosphate buffered saline with 0.05% TWEEN® 20 (PBST) for 1 hr followed by three 5 min washes with PBST. The blots will then be probed for 1 hr with 1:50,000 dilutions of each of the rabbit sera obtained against each of the seven proteins prepared in 3% skim milk respectively. After probing with the primary antibody, the blots will be washed three times with PBST and probed with 1:25,000 dilution of affinity purified antibody peroxidase labeled goat anti-rabbit IgG, H+L for 1 hr. The blots will be finally washed with PBST and stained with TMB membrane peroxidase substrate till the chemiluminescent immuno-reactive spots appeared on the blot. The blots will be dried and the images will be captured through ChemiDoc XRS system (BIO-RAD®, Inc. USA).

### EXAMPLE 7

### Confirmation of the Protein Candidates

### ProteinChip PG20 Array Technique

In order to identify the 7 B. anthracis signature proteins, ProteinChip PG20 arrays were used. Each ProteinChip PG20 array contains 8 spots and 12 arrays that could be processed in a standard 8×12 format. Parallel processing of the 8×12 cassette was done to allow simultaneous screening of all the sera samples at the same time. The procedure comprises of coupling antibody to the ProteinChip array. Only 5 μl of each of the rabbit serum sample was added to PG20 array spot and incubated in a humidity chamber at room temperature for an hour, followed by placing the array in a bioprocessor to wash the chips 2×5 minutes with 100 μl phosphate buffer saline with TWEEN® 80 (“PBST”)+0.5 M NaCl and further wash 2×5 minutes with 100 μl phosphate buffer saline (“PBS”) (ICI Americas Inc. Wilmington, Del.). The ProteinChip arrays were then treated with 90 μl of PBS and 10 μl B. anthracis spore lysate to capture the B. anthracis antigens on to the chip by incubating for one hour at room temperature. The arrays were washed stringently to reduce the non-specific binding of proteins to the surface (1×5 minutes with 150 μl PBST, 2×5 minutes with 150 μl PBS). Further, the arrays were rinsed twice with 200 μl volumes of 1 mM NaHepes and the spots were air-dried. Energy absorbing molecule (“EAM”) was prepared using 200 μl each of 99.8% acetonitrile and 1.0% TFA and then added into the vial containing ProteinChip sinapinic acid EAM powder to obtain a final concentration of 25 mg/ml. The vial was vortexed for 5 minutes to dissolve the EAM powder. Two 1 μL aliquots of this 50% saturated EAM solution was then added to each spot on the array and air-dried completely (approximately 10 minutes) before reading in a ProteinChip SELDI reader. After application of EAM, the arrays were inserted in a SELDI-TOF-MS for rapid acquisition of both low and high laser energy spectra spanning the 1 kD-300 kD mass range.

### Data Acquisition and Analysis

The ProteinChip SELDI reader has a raster-scanning feature that allows precise partitioning of the spot surface. This allows for multiple acquisition protocols to be carried out from the same spot, while each protocol samples an even distribution of the entire spot surface. To optimize different mass ranges, spectra spanning the mass ranges of 1 kD-10 kD, 10 kD-50 kD and 50 kD-300 kD were collected using laser settings of 1500 nJ, 3000 nJ and 5000 nJ respectively. The ProteinChip reader used in this study have been regularly tested using Bio-Rad's SELDI-MS IQ/OQ kit, testing for detector calibration, detector sensitivity and mass accuracy.

ProteinChip Data Manager software was utilized. Spectra were first sorted according to the varying laser energies used. Proper baseline fit and external mass calibration using Bio-Rad all-in-one peptide and protein standards was performed. The software's Expression Difference Mapping (EDM) feature was used to identify markers that were differentially expressed across samples groups (i.e. control sera vs. infected sera). Univariate analysis of the varying peak intensities provided statistical (p-value) assignment of those B. anthracis protein markers with the highest selective potential. The seven selected protein candidates of B. anthracis spore was confirmed using ProteinChip antibody array coupled with SELDI-TOF-MS.

TABLE 4
Identification of conserved signature proteins of B. anthracis 34F2 by ProteinChip
antibody array technology.

| Rabbit Sub-group | | 3000 nJ | Sample | Chip barcode/spot/partition |
|---|---|---|---|---|
| m/z | Peak Intensity | S/N | name | Spectrum |
| [symbol] | [symbol] | [symbol] | [symbol] | [symbol] |
| [symbol] | [symbol] | [symbol] | [symbol] | [symbol] |
| 36392.4547 | 0.052249646 | 4.363582753 | MetN | 1070133540_spotA_2.3 |
| 37215.83117 | 0.047455583 | 4.70777819 | RANS | 1070133120_spotA_2.3 (neg. |
| | | | | control) |
| [symbol] | [symbol] | [symbol] | [symbol] | [symbol] |
| 75072.36777 | 0.017384129 | 5.352235559 | RANS | 1070133120_spotA_2.3 |
| | | | | (neg. control) |
| [symbol] | [symbol] | [symbol] | [symbol] | [symbol] |
| [symbol] | [symbol] | [symbol] | [symbol] | [symbol] |
| 94652.27615 | 0.021542899 | 6.717564947 | RANS | 1070133120_spotA_2.3 |
| | | | | (neg. control) |

[symbol]

indicates data missing or illegible when filed

### Immunogold Labeling of Bacillus Spores

Bacillus spores were pelleted in microcentrifuged and enrobed in 2.5% Low Melting Temperature (“LMT”) agarose. Agarose blocks containing spores were trimmed into ˜1 mm³ size and dehydrated in 30% ethanol at room temperature, followed by 50% ethanol at 4° C. and 70%, 90% and 100% ethanol at −20° C. (15 min each). Post-fix staining with 1% uranyl acetate in 70% ethanol was performed after the 70% ethanol dehydration step at −20° C. for 1 hr. After two changes of 100% ethanol, spore containing agarose blocks were infiltrated with Unicryl acrylic resin (BB International) by immersing sequentially in a 1:1, 2:1 unicryl and 100% ethanol mixture and 100% unicryl, 1 hr each. After additional 100% unicryl incubation, agarose blocks were left in 100% unicryl at −20° C. overnight before polymerization at −20° C. under UV for 48 hours. Embedded spores were then ultra-thin sectioned at ˜90 nm thickness (Ultracut E, Reichert-Jung) and collected onto formvar coated Nickel grids. Immunogold labeling of spore sections were performed as follows. Grids were inverted section-side facing down onto a drop of blocking solution containing 1% BSA,1% fish gelatin, 0.01M glycine in PBS, pH 7.4 for 10 min and transferred onto 10 μl droplet of primary rabbit (Crd,RhsA,FusA at 1:100; AcnA,Dal-1,Eag at 1:50; MetN at 1:20) sera diluted in blocking solution for 30min at room temperature. Grids were then washed five times by transferring onto five 30 μl rinse solutions (0.1% BSA, 1% Fish gelatin in PBS pH 7.4) consecutively. Labeling with 10 nm-gold conjugated Protein G or goat anti-rabbit secondary antibody were performed the same way and followed with five washes. Grids were then fixed with (2% glutaraldehyde in PBS) 5 min, rinsed with water and air dry. Grids were examined using a Technai T12 transmission electron microscope (FEI) at 80 keV and images were acquired with an AMT digital camera. The seven selected protein candidate were successfully located in the core or the exosporium of B. anthracis 34F2 spores.

Having described the invention, one of skill in the art will appreciate in the appended claims that many modifications and variations of the present invention are possible in light of the above teachings. It is therefore, to be understood that, within the scope of the appended claims, the invention may be practiced otherwise than as specifically described.

### REFERENCES

1. Sellman, B., Mourez, M., Collier, R. 2001. Dominant-negative mutants of a toxin subunit: An approach to therapy of anthrax. Science 292: 695-7.
2. Petosa, D., Collier, R., Klimpel, K., Leppla, S., Liddington, R. 1997. Crystal structure of the anthrax toxin protective antigen. Nature 385: 833-8.
3. Little, S. F., Novak, J., Lowe, J., Singh, Y., Klimpel, K. R., Lingerding, B. C., and Friedlander, A. M. 1996. Characterization of lethal factor binding and cell receptor binding domains of protective antigen of Bacillus anthracis protective antigen. Microbiology 142: 707-15.
4. Brossier, F., Sirard, J-C., Guidi-Rontani, C., Duflot, E., Mock., M. 1999. Functional analysis of the carboxy-terminal domain of Bacillus anthracis protective antigen. Infect. Immun. 67: 964-7.
5. Brossier, F., Weber-Levy, M., Mock, M., Sirard, J. 2000. Role of toxin functional domains in anthrax pathogenesis. Infect. Immun. 68: 1781-6.
6. Mogridge, J., Moourez, M., Collier, R. 2001. Involvement of domain 3 in oligomerization by the protective antigen moiety of anthrax toxin. J. Bacteriol. 183: 2111-6.
7. Brachman, P., Gold, H., Plotkin, S., Fekety, F., Werrin, M., Ingraham, N. 1962. Field evaluation of a human anthrax vaccine. Am. J. Public Health 52: 632-45.
8. Fellows, P., Linscott, M., Ivins, B., Pitt, M. L. M., Rossi, C. A., Gibbs, P. H., and Friedlander, A. M. 2001. Efficacy of a human anthrax vaccine in guinea pigs, rabbits, and rhesus macaques against challenge by Bacillus anthracis isolates of diverse geographical origin. Vaccine 19: 3241-7.
9. Tumbull, P., Broster, M., Carman, J., Manchee, R., Melling, J. 1986. Development of antibodies to protective antigen and lethal factor components of anthrax toxin in humans and guinea pigs and their relevance to protective immunity. Infect Immun. 52: 356-63.
10. Ariel N., Zvi A., Makarova K S, Chitlaru T., Elhanany E., et al (2003) Genome-based bioinformatic selection of chromosomal Bacillus anthracis putative vaccine candidates coupled with proteomic identification of surface-associated antigens. Infect Immun 71(8): 4563-4579.
11. Read T D, Peterson S N, Tourasse N, Baillie L W, Paulsen I T, et al (2003) the genome sequence of Bacillus anthracis Ames and comparison to closely related bacteria. Nature 423:81-86.

What we claim is:

1. An immunogenic composition for the induction of an immune response against Bacillus cereus group bacteria comprising one or more purified and isolated proteins, or immunogenic fragments thereof, selected from the group consisting of cell surface protein, gram positive anchor, conserved repeat domain protein, transcription-repair coupling factor, pyruvate decarboxylase, predicted exporter, aconitate hydratase 1, S-layer protein EA1 precursor, polyribonucleotide nucleotidyl transferase, translation elonation factor G, pyruvate kinase, CTP synthase, chaperonin, 60 kDa, Δ-1-pyrroline-5 carboxylate dehydrogenase, aldehyde dehydrogenase, alanine racemase, phosphoglycerate kinase, pyruvate dehydrogenase complex E1 component, alpha subunit and ABC transporter, ATP binding protein and a combination thereof.

2. The composition of claim 1, wherein said proteins are recombinant.

3. A method of inducing an immune response against Bacillus cereus group bacteria comprising administering the proteins of claim 1.

4. The method of claim 3, wherein said composition is recombinant.

5. The method of claim 4, wherein said composition is expressed in a live attenuated bacterial vector.

6. The method of claim 4, wherein said composition is encoded by a viral vector.

7. A method for detecting Bacillus cereus group bacteria, comprising:
a. exposing patient sera to one or more proteins or immunogenic fragments thereof, selected from the group consisting of cell surface protein, gram positive anchor, conserved repeat domain protein, transcription-repair coupling factor, pyruvate decarboxylase, predicted exporter, aconitate hydratase 1, S-layer protein EA1 precursor, polyribonucleotide nucleotidyl transferase, translation elonation factor G, pyruvate kinase, CTP synthase, chaperonin, 60 kDa, Δ-1-pyrroline-5 carboxylate dehydrogenase, aldehyde dehydrogenase, alanine racemase, phosphoglycerate kinase, pyruvate dehydrogenase complex E1 component, alpha subunit and ABC transporter, ATP binding protein and a combination thereof, and
b. measuring bound antibody.

8. The method of claim 7, wherein said bound antibody is detected using ELISA or microarray.

9. A method for detecting Bacillus cereus group bacteria in a sample, comprising:
a. incubating said sample with one or more templates, whereby a PCR reaction takes place which amplifies B. cereus nucleic acids to produce one or more B. cereus amplification products; and
b. detecting the presence of said B. cereus amplification products by Southern hybridization, wherein the presence of said amplification products is indicative of the presence of B. cereus in the sample.

10. The method of claim 9, wherein said template is a protein or immunogenic fragments of said protein selected from the group consisting of cell surface protein, gram positive anchor, conserved repeat domain protein, transcription-repair coupling factor, pyruvate decarboxylase, predicted exporter, aconitate hydratase 1, S-layer protein EA1 precursor, polyribonucleotide nucleotidyl transferase, translation elonation factor G, pyruvate kinase, CTP synthase, chaperonin, 60 kDa, Δ-1-pyrroline-5 carboxylate dehydrogenase, aldehyde dehydrogenase, alanine racemase, phosphoglycerate kinase, pyruvate dehydrogenase complex E1 component, alpha subunit and ABC transporter, ATP binding protein and a combination thereof.

11. The method of claim 9, wherein said detection step further comprising exposing said amplified products to one or more polynucleotide sequences immobilized on a microchip, wherein said polynucleotide sequences encode the entire or a immunogenic fragment of a protein selected from the group consisting of cell surface protein, gram positive anchor, conserved repeat domain protein, transcription-repair coupling factor, pyruvate decarboxylase, predicted exporter, aconitate hydratase 1, S-layer protein EA1 precursor, polyribonucleotide nucleotidyl transferase, translation elonation factor G, pyruvate kinase, CTP synthase, chaperonin, 60 kDa, A-1-pyrroline-5 carboxylate dehydrogenase, aldehyde dehydrogenase, alanine racemase, phosphoglycerate kinase, pyruvate dehydrogenase complex E1 component, alpha subunit and ABC transporter, ATP binding protein and a combination thereof.
